(19) United States
(12) Patent Application Publication
(10) Pub. No.: US 2005/0009020 A1
(43) Pub. Date: Jan. 13, 2005

(54) Microarray method for enriching dna fragments from complex mixtures

(76) Inventor: Jurgen Distler, Berlin (DE)

Correspondence Address: KRIEGSMAN & KRIEGSMAN, 665 FRANKLIN STREET, FRAMINGHAM, MA, 01702, US

(21) Appl. No.: 10/474,779

(22) PCT Filed: Apr. 11, 2002
(86) PCT No.: PCT/EP02/04056

(30) Foreign Application Priority Data
Apr. 12, 2001 (DE) 101-19-468.4

(51) Int. Cl. C12Q 1/68; C12P 19/34

(52) U.S. Cl. 435/6; 435/91.2

(57) ABSTRACT

A method for the simultaneous, parallel, selective enrichment of different DNA segments which are obtained from different tissues by complex amplifications and have desired individual sequence properties is described. These properties of the DNA fragments, particularly the presence of 5-methylcytosines, can be identified by hybridization on DNA microarrays. The desired DNA fragments are enriched by several repetitions of the operating steps (hybridization, dehybridization and reamplification). The method combines the SELEX method with complex DNA arrays, which are used for the enrichment of DNA fragments. The sequence of the amplificates is then analyzed.

The present invention describes a method for the selective enrichment of DNA segments with desired sequence properties from complex DNA mixtures, which have been produced by amplifications. These enriched DNA fragments obtained by the present invention can then be analyzed in detail by standard methods.

The polymerase chain reaction (PCR) is a method by means of which, in principle, any DNA can be selectively amplified. This method comprises the use of a set of at most two oligonucleotides with predefined sequence, so-called primers, which hybridize to DNA strands that are complementary to them and define the boundaries of the sequence to be amplified.

The oligonucleotides initiate the DNA synthesis, which is catalyzed by a heat-stable DNA polymerase. A melting step and a re-annealing step typically are provided in each round of synthesis. This technique permits amplification of a given DNA sequence by several orders of magnitude in less than one hour.

PCR has gained a wide acceptance due to the simplicity and reproducibility of these reactions. For example, PCR is used for the diagnosis of hereditary malfunctions and when such disorders are suspected.

PCR reactions are also known, which use more than two different primers. They primarily serve for simultaneous amplification in one vessel of several fragments, also with base sequences that are known at least for the most part. In this case also, the primers used specifically bind to certain segments of the template DNA. In such cases, one speaks of “multiplex PCR”, which primarily only has the objective of being able to simultaneously amplify several specific fragments and thus to save on materials and experimental effort.

Often, however, an amplification of a given sample is also conducted simply to propagate the material for a subsequent investigation. The sample to be investigated is first amplified in this case, either starting from genomic DNA or RNA. For the most part, it is necessary to label at least one of the primers, e.g., with a fluorescent dye, in order to be able to identify the fragment in subsequent experiments.

This amplified DNA is utilized for the identification of mutations and polymorphisms. The following analytical methods are considered for this: e.g., the primer extension reaction, sequencing according to Sanger, or, e.g., restriction digestion and subsequent investigation on agarose gels, for example, and hybridization on DNA microarrays.

While the investigation of sample DNA with primer oligonucleotides of predetermined sequence is prior art, a method is lacking which makes possible the purification and identification of DNA fragments with desired sequence properties from complex mixtures of DNA molecules, such as are obtained by multiplex PCR or random PCR reactions.

Complex PCR amplifications, e.g., “whole genome amplifications” (random PCR) are used for the simultaneous propagation of a plurality of fragments of DNA samples. The highly diverse fragments that are obtained may be used, among other things, for genotyping, mutation analysis and related subject fields.

An overview of the state of the art in oligomer array production can be derived from a special issue of Nature Genetics which appeared in January 1999 (Nature Genetics Supplement, Volume 21, January 1999), the literature cited therein, and U.S. Pat. No. 5,994,065 on methods for the production of solid supports for target molecules such as oligonucleotides. In addition to deoxyribonucleic acids (DNA), peptide nucleic acids (PNA) or locked nucleic acids (LNA) can also be fixed on the surface of oligomer arrays.

Peptide nucleic acids (PNA) (Nielsen, P. E., Buchardt, O., Egholm, M. and Berg, R. H. 1993. Peptide nucleic acids. U.S. Pat. No. 5,539,082; Buchardt, O., Egholm, M., Berg, R. H. and Nielsen, P. E. 1993. Peptide nucleic acids and their potential applications in biotechnology. Trends in Biotechnology, 11: 384-386) have an uncharged backbone, which simultaneously deviates chemically very greatly from the familiar sugar-phosphate structure of the backbone in nucleic acids. The backbone of a PNA has an amide sequence instead of the sugar-phosphate backbone of common DNA. PNA hybridizes very well with DNA of complementary sequence. The melting temperature of a PNA/DNA hybrid is higher than that of the corresponding DNA/DNA hybrid and the dependence of hybridization on buffer salts is relatively small.

Locked nucleic acids (LNA) (Nielson et al. (1997 J. Chem. Soc. Perkin Trans. 1, 3423); Koshkin et al. (1998 Tetrahedron Letters 39, 4381); Singh & Wengel (1998 Chem. Commun. 1247); Singh et al. (1998 Chem. Commun. 455)) have built-in “internal bridges”. The synthesis of LNAs and their properties have been described by numerous authors. Thus, like PNAs, LNAs have a greater thermal stability in pairing with DNA than conventional DNA/DNA hybrids.

5-Methylcytosine is the most frequent covalently modified base in the DNA of eukaryotic cells. For example, it plays a role, in the regulation of transcription, in genetic imprinting and in tumorigenesis. The identification of 5-methylcytosine as a component of genetic information is thus of considerable interest. 5-Methylcytosine positions, however, cannot be identified by sequencing, since 5-methylcytosine has the same base-pairing behavior as cytosine. In addition, in the case of a PCR amplification, the epigenetic information which is borne by the 5-methylcytosines is completely lost.

A relatively new method that in the meantime has become the most widely used method for investigating DNA for 5-methylcytosine is based on the specific reaction of bisulfite with cytosine, which, after subsequent alkaline hydrolysis, is then converted to uracil, which corresponds in its base-pairing behavior to thymidine. In contrast, 5-methylcytosine is not modified under these conditions. Thus, the original DNA is converted so that methylcytosine, which originally cannot be distinguished from cytosine by its hybridization behavior, can now be detected by “standard” molecular biology techniques as the only remaining cytosine, for example, by amplification and hybridization or sequencing. All of these techniques are based on base pairing, which is now fully utilized.

The prior art which concerns sensitivity is defined by a method that incorporates the DNA to be investigated in an agarose matrix, so that the diffusion and renaturation of the DNA is prevented (bisulfite reacts only on single-stranded DNA) and all precipitation and purification steps are replaced by rapid dialysis (Olek A, Oswald J, Walter J. A modified and improved method for bisulphite based cytosine methylation analysis. Nucleic Acids Res. 1996 Dec. 15;24(24 ): 5064-6). Individual cells can be investigated by this method, which illustrates the potential of the method. Of course, up until now, only individual regions of up to approximately 3000 base pairs long have been investigated; a global investigation of cells for thousands of possible methylation analyses is not possible. Of course, this method also cannot reliably analyze very small fragments of small quantities of sample. These are lost despite the protection from diffusion through the matrix.

An overview of other known possibilities for detecting 5-methylcytosines can be derived from the following review article: Rein T, DePamphilis M L, Zorbas H. Identifying 5-methylcytosine and related modifications in DNA genomes. Nucleic Acids Res. 1998 May 15; 26(10): 2255-64.

The bisulfite technique has been previously applied only in research, with a few exceptions (e.g., Zeschnigk M, Lich C, Buiting K, Doerfler W, Horsthemke B. A single-tube PCR test for the diagnosis of Angelman and Prader-Willi syndrome based an allelic methylation differences at the SNRPN locus. Eur J Hum Genet. 1997 March-April; 5(2):94-8). However, short, specific segments of a known gene have always been amplified after a bisulfite treatment and either completely sequenced (Olek A, Walter J. The pre-implantation ontogeny of the H19 methylation imprint. Nat Genet. 1997 November; 17(3): 275-6) or individual cytosine positions have been detected by a “primer extension reaction” (Gonzalgo M L, Jones P A. Rapid quantification of methylation differences at specific sites using methylation-sensitive single nucleotide primer extension (Ms-SNuPE). Nucleic Acids Res. 1997 Jun. 15; 25(12): 2529-31, WO Patent 95-00669) or an enzyme step (Xiong Z, Laird PW. COBRA: a sensitive and quantitative DNA methylation assay. Nucleic Acids Res. 1997 Jun. 15; 25(12): 25324). Detection by hybridization on DNA microarrays has also been described (Olek et al., WO 99-28498).

To analyze PCR products, they can be provided, e.g., with a fluorescent label or a radioactive label. These labels can be introduced either on the primers or on the nucleotides. Particularly suitable for fluorescent labels is the simple introduction of Cy3 and Cy5 dyes at the 5′-end of the respective primer. The following are also considered as fluorescent dyes: 6-carboxyfluorescein (FAM), hexachloro-6-carboxyfluorescein (HEX), 6-carboxy-x-rhodamine (ROX) or tetrachloro-6-carboxyfluorescein (TET).

As shown, it is common at the present time, for identifying cytosine methylations, to treat the DNA samples with bisulfite and to use them subsequently for identifying primer oligonucleotides of known sequence. A plurality of SELEX methods are described for the enrichment of specific DNA fragments (e.g., U.S. Pat. No. 5,270,163; U.S. Pat. No. 6,238,927; U.S. Pat. No. 5,288,609. A method is lacking, however, for the selective enrichment of DNA segments from complex DNA molecule mixtures, which combines the advantages of the highly parallel analysis possibilities of DNA arrays with a SELEX method.

A method will be provided that makes it possible to simultaneously and efficiently enrich several DNA fragments which have been obtained from different tissues by PCR reactions and which possess a desired sequence property. The desired DNA fragments will be enriched by hybridization (preferably by complex DNA arrays), dehybridization (preferably by selected regions of the DNA arrays) and reamplification of the dehybridized DNA fragments, whereby the desired DNA fragments will be enriched by multiple repetition of the operating steps (hybridization, dehybridization and reamplification). The complexity of the DNA array in the enrichment process will be reduced in the preferred method. The advantage of the method will lie in the fact that unknown DNA fragments can be identified, which have been produced by complex amplifications (multiplex und random PCR), in which the primers that are used are known, but the DNA fragment mixture that is produced is unknown. The desired properties of the sought DNA fragments can be identified, particularly the presence of 5-methylcytosines, by hybridization, primarily on oligonucleotide microarrays.

A method will be described for the selective enrichment of individual specific PCR fragments from complex fragment mixtures, which have been produced by complex PCR amplifications. The fragment enrichment is based on the hybridization properties of the individual fragments, which are preferably analyzed with oligonucleotide arrays. In detail, the method is comprised of the following steps:

1. The DNA segments are produced and simultaneously labeled by amplification methods that produce complex mixtures of amplificates, preferably in the presence of a heat-stable DNA polymerase. The fragments are preferably produced by multiplex PCR reactions or random PCR reactions.

The labeling of the amplification products for the subsequent hybridization experiments can be produced preferably by the use of primer oligonucleotides in the PCR reaction, which are preferably labeled with fluorescent dyes with different emission spectra (e.g., Cy3, Cy5, FAM, HEX, TET or ROX) or with fluorescent dye combinations in the case of primers labeled by energy-transfer fluorescent dye.

The labeling of the primer oligonucleotides may also be carried out particularly with radionuclides or preferably with removable mass labels which are detected in a mass spectrometer. Molecules, which only produce a signal in a further chemical reaction, may also be preferably used for labeling.

The labeling of the PCR products may also be preferably produced by DNA nucleotide building blocks, which are fluorescently labeled or labeled with radionucleotides and which are employed in the PCR reactions.

The required nucleic acids that serve as a template for the PCR reactions are preferably obtained from a genomic DNA sample, whereby sources for DNA include, e.g., cell lines, blood, sputum, stool, urine, cerebrospinal fluid, tissue embedded in paraffin, for example, tissue from eyes, intestine, kidney, brain, heart, prostate, lung, breast or liver, histological slides and all possible combinations thereof.

These nucleic acids can preferably be chemically treated.

The chemical treatment preferably comprises embedding the DNA in agarose and subsequently reacting the nucleic acid sample with a bisulfite solution (=disulfite, hydrogen sulfite), whereby 5-methylcytosine remains unchanged and cytosine is converted to uracil or another base similar to uracil in its base-pairing behavior. In the chemical treatment, a reagent that denatures the DNA duplex and/or a radical trap can also be preferably employed.

As a template for an RT-PCR, RNA preparations of different cells and tissues may also be used, e.g., cell lines, blood, sputum, stool, urine, cerebrospinal fluid, tissue embedded in paraffin, for example, tissue from eyes, intestine, kidney, brain, heart, prostate, lung, breast or liver, histological slides and all combinations thereof, which are converted to DNA with reverse transcription.

The fragments may alternatively be labeled for the described procedure preferably also after PCR amplification, by known molecular biology methods.

2. The complex mixture of labeled DNA fragments, which were produced by a complex PCR amplification, which is described under Item 1, will preferably hybridize to oligomer arrays (screening arrays), which bear different oligonucleotides, PNA oligomers or LNA oligomers.

The oligonucleotides, PNA oligomers or LNA oligomers are preferably arranged on the solid phase in the form of a rectangular or hexagonal grid.

The labels introduced on the amplificates can preferably be identified at any position of the solid phase, on which an oligonucleotide sequence is found, as long as a hybridization has occurred at this position.

3. For the enrichment of desired DNA fragments, first the DNA fragments, which are reversibly bound to the oligomer array by the oligonucleotides by means of a hybridization event, will be stripped off by a dehybridizing step.

In a preferred method, the dehybridization of the entire oligomer array will not be conducted in one step. but selected partial regions of the oligomer array will be subjected to separate dehybridization steps.

The PCR fragments obtained in this way serve as the template for a second PCR reaction, which is conducted with the reaction conditions of the first PCR reaction, which produced the original complex mixture of DNA fragments.

4. For the enrichment of desired DNA fragments, the following additional method steps are conducted:

4.1. The amplificates of this second PCR reaction are hybridized to a new oligomer array (identification array). This identification array bears oligonucleotides, PNA oligomers or LNA oligomers which have hybridized with the original fragment mixture in the desired way. The number of oligonucleotides, PNA oligomers or LNA oligomers on the identification array is significantly smaller when compared to the screening array.

4.2 The DNA fragments, which are reversibly bound to the identification array by the oligonucleotides by means of a hybridization event, will be stripped off by a dehybridizing step.

4.3 Since too many DNA fragments have still been produced in the second PCR amplification, the method steps 4.1 and 4.2 will be repeated several times, preferably 2 to 5 times. For example, the dehybridized fragments are the template of a third PCR reaction. The reaction conditions of this PCR are also identical with the conditions of the first PCR amplification. In these repeated steps, the following can be employed: a) identical identification arrays can be used, b) identification arrays with a reduced number of bound oligonucleotides can be used, or c) stepwise more selective conditions can be selected for the dehybridization (see 4.2).

5. The amplificates of the last PCR can now be identified with known molecular biology methods (e.g., cloning and DNA sequencing), due to the correspondingly reduced complexity of the different amplificates. It is possible with this method to enrich DNA fragments with desired sequence properties (DNA and RNA target identification), if they can always be identified by hybridization. It is thus particularly suitable for the enrichment of DNA fragments which show differences in methylation at defined CpG positions. DNA fragments which bear defined SNPs can also be enriched with this method. If RNA is used as the template for the first PCR, e.g., DNA fragments from genes that are selectively transcribed in certain tissues can be enriched. This method is particularly suitable for identifying different tissue-specific and/or disease-specific MESTs (methylated sequence tags) or SNPs (single nucleotide polymorphisms) in a highly parallel method.

Simultaneously, this method can be employed for constructing a screening assay, which makes it possible to identify target sites for pharmaceutical active ingredients, which, e.g., participate in DNA methylation or RNA transcription.

The identification of genes which are diagnostically relevant for the following disorders is a decisive factor in determining target sites for pharmaceutical active ingredients and the development of new medications against these: cancer diseases; CNS malfunctions, damage or disease; symptoms of aggression or behavioral disturbances; clinical, psychological and social consequences of brain damage; psychotic disturbances and personality disorders; dementia and/or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as a consequence of an abnormality in the development process; malfunction, damage or disorder of the skin, the muscles, the connective tissue or the bones; endocrine and metabolic malfunction, damage or disease; headaches or sexual malfunction.

The method is preferably used for distinguishing cell types or tissues or for investigating cell differentiation.

The following examples explain the invention:

### EXAMPLE 1:

### Preparation of a Complex Mixture of DNA Fragments by PCR Amplification for a DNA Methylation Analysis

In the first step, genomic DNA from healthy control tissue and a tumor specimen are isolated with the DNA Extraction Kit (Stratagene, La Jolla, [Calif.], U.S.A.) and are treated with the use of bisulfite (hydrogen sulfite, disulfite) in such a way that all of the unmethylated cytosines at the 5-position of the base are modified such that a base that is different in its base-pairing behavior is formed, while the cytosines that are methylated in the 5-position remain unchanged. If bisulfite is used for the reaction, then an addition occurs on the unmethylated cytosine bases. Also, a denaturing reagent or solvent as well as a radical trap must be present. A subsequent alkaline hydrolysis then leads to the conversion of unmethylated cytosine nucleobases to uracil. This converted DNA serves for the detection of methylated cytosines.

In the second step of the method, the treated DNA sample is diluted with water or an aqueous solution. A desulfonation of the DNA (10-30 min, 90-100° C.) at alkaline pH is then preferably conducted.

In the third step of the method, the DNA fragments are amplified in a polymerase chain reaction with a heat-stable DNA polymerase. In the present case, a complex mixture of labeled DNA fragments is prepared by PCR from the bisulfite-treated DNA preparations of the control tissue and the tumor specimen. For this purpose, the two DNA preparations are employed in a PCR reaction with 128 oligonucleotide primer pairs, half of which are labeled with the fluorescent dye Cy5. In this PCR, a mixture of at least 64 DNA fragments with a length of about 200-950 base pairs is produced. These amplificates serve as samples, which hybridize to oligonucleotides (oligonucleotide array) previously bound to a solid phase, with the formation of a duplex structure. The detection of the hybridization product is based on primer oligonucleotides fluorescently labeled with Cy5, which were used for the amplification. A hybridization reaction between the amplified DNA and this oligonucleotide occurs only if a methylated cytosine has been present in the bisulfite-treated DNA, e.g., in the sequence context GGATTTAGCGGTAAGTAT. Thus the methylation state of the respective cytosine to be investigated decides the hybridization product.

In the fourth step of the method, the DNA fragments amplified from the two DNA preparations are each hybridized with an oligonucleotide array to which 500-2048 oligonucleotides have been bound, and the fluorescent signals are quantitatively analyzed with a commercially available chip scanner (Genepix 4000, Axon Instruments).

### EXAMPLE 2:

### Identification of DNA Fragments with Different Methylation State

Oligonucleotides which displayed hybridization signals after the hybridization (see Example 1) with DNA fragments amplified from the control tissue, in contrast to amplificates from the tumor tissue, were used for the preparation of a new oligonucleotide array (identification array).

The following steps were then conducted in the method:
1) Bisulfite-treated DNA from the control tissue was used as the template for a PCR amplification (see Example 1, Item 3)
2) The identification array was hybridized with the amplificates from the control tissue and
3) then the DNA fragments bound to the array by hybridization were isolated by a dehybridization of the identification array, preferably by an incubation with water at 75° C.
4) These DNA fragments were utilized in a PCR reaction with the same 128 oligonucleotide primer pairs (see Example 1, Item 3). The agarose gel analysis of this PCR reaction showed a DNA fragment mixture, which had a reduced complexity in comparison to Example 1.
5) The fragments obtained by the dehybridization of the identification array served as the template for a PCR reaction with the 128 oligonucleotide primer pairs (see Example 1, Item 3).
6) The PCR amplificates from Item 5) were again hybridized with the identification array.

Steps 3-6 were repeated until only individual DNA fragments could be identified in the agarose gel analysis. These fragments could then be analyzed with known methods (e.g., cloning and sequencing).

### EXAPMLE 3:

Enrichment of a DNA Fragment with Different Methylation State in Two Tissues.

Different DNA fragments (up to 1000) were produced from bisulfite-treated DNA from control tissue and tumor tissue by PCR with degenerate, Cy5-labeled primers. Each of these PCR products from the control tissue and from the tumor tissue was hybridized separately, depending on the tissue type, with a DNA array. Immobilized on the DNA array were 2000 pairs of oligonucleotides (with the general sequences NNNNNNNNCGNNNNNNNN and NNNNNNNNTGNNNNNNNN), which hybridize either with one or several PCR products if a methylated cytosine was present in the corresponding bisulfite-treated DNA, e.g., in the sequence context GGATTTAGCGGTAATAT, or hybridize if an unmethylated cytosine was present in the corresponding bisulfite-treated DNA, e.g., in the sequence context GGATTTAGTGGTAATAT.

After the hybridization, the fluorescent signals were quantitatively analyzed with a commercially available chip scanner (Genepix 4000, Axon Instruments). A comparison of the array hybridized with PCR products from control and tumor tissues made possible the identification of oligonucleotides which hybridized with PCR products which have a different methylation state in the two tissues.

The DNA array was divided with a perforated mask into 32 fields for the enrichment, whereby 32 individual dehybridizations could be conducted on 120 oligonucleotides (see Example 2). In this way, 32 DNA fragment pools were prepared. Since the position of the oligonucleotides which revealed a difference in methylation between the samples was known, one of the 32 DNA fragment pools served as the template for the second PCR. This PCR was conducted with the same primer as the first PCR.

The PCR fragments from the second PCR were now hybridized with a DNA array (identification array), which bore only the 120 oligonucleotides of the corresponding dehybridization pool. After dehybridization of this identification array, which can be conducted again in a position-specific manner if needed, with the help of the perforated mask, the dehybridized fragments served as the template for the third PCR. By analogy to Example 2, PCR, hybridization and dehybridization were repeated until individual DNA fragments could be identified in the agarose gel analysis. These fragments could then be analyzed with known methods (e.g., cloning and sequencing).

1. A method for the parallel selective enrichment of many individual, specific PCR fragments from complex fragment mixtures, hereby characterized in that the following steps are conducted:
a) the DNA segments are produced by amplification methods that produce complex mixtures of amplificates and thus are simultaneously labeled;
b) the amplificates will hybridize to oligomer arrays which bear different oligonucleotides;
c) the PCR amplificates hybridized to the oligomer arrays are stripped from the oligonucleotides and serve as the template for a repeated PCR amplification and subsequent hybridization corresponding to steps a) and b);
d) step c) is repeated several times; whereby the complexity of the array for each repetition of step c) is reduced.
e) the amplificates are identified.

2. The method according to claim 1 [further characterized] in that the amplificates in step c) are stripped from the entire array or from selected partial regions of the array.

3. The method according to claim 1, further characterized in that the PCR amplification methods in step a) are either multiplex PCR reactions or random PCR reactions.

4. The method according to claim 1, further characterized in that in step a), the DNA segments to be amplified are chemically treated.

5. The method according to claim 1, further characterized in that in step a), the nucleic acid sample is repeatedly chemically reacted with a reagent, whereby 5-methylcytosine remains unchanged and cytosine is converted to uracil or another base similar to uracil in its base-[pairing] behavior.

6. The method according to claim 5, further characterized in that the reagent involves a bisulfite (=hydrogen sulfite, disulfite).

7. The method according to one of claims 4, 5 or 6, further characterized in that the chemical treatment is conducted after embedding the DNA in agarose.

8. The method according to one of claims 4, 5 or 6, further characterized in that in the chemical treatment, a reagent that denatures the DNA duplex and/or a radical trap is present.

9. The method according to claim 1, further characterized in that in step a) the segments to be amplified are comprised of RNA and are converted to DNA with reverse transcription.

10. The method according to claim 1, further characterized in that the oligonucleotides in steps b) and c) involve DNA, PNA or LNA oligomers.

11. The method according to claim 1, further characterized in that in steps b) and c) the fragments are alternatively labeled after the PCR amplification or after the reamplification.

12. The method according to claim 1, further characterized in that the PCR amplifications are conducted in the presence of a heat-stable polymerase.

13. The method according to one of the preceding claims, further characterized in that the labeling of the primer oligonucleotides or DNA nucleotide building blocks involves fluorescent dyes with different emission spectra (e.g., Cy3, Cy5, FAM, HEX, TET or ROX) or fluorescent dye combinations in the case of primer oligonucleotides or DNA nucleotide building blocks labeled by energy-transfer fluorescent dye.

14. The method according to one of the preceding claims, further characterized in that the labels are radionuclides.

15. The method according to one of the preceding claims, further characterized in that the labels are removable mass labels which are detected in a mass spectrometer.

16. The method according to one of the preceding claims, further characterized in that molecules that only produce a signal in a further chemical reaction are used for the labeling.

17. The method according to one of the preceding claims, further characterized in that the oligonucleotides are arranged on a solid phase in the form of a rectangular or hexagonal grid.

18. The method according to one of the preceding claims, further characterized in that the labels that are introduced on the amplificates at each position of the solid phase at which an oligonucleotide sequence is found can be identified.

19. The method according to one of the preceding claims, wherein the DNA segments or RNA samples that are converted into DNA with reverse transcription were obtained from a genomic sample, whereby sources for DNA or RNA include, e.g., cell lines, blood, sputum, stool, urine, cerebrospinal fluid, tissue embedded in paraffin, for example, tissue from eyes, intestine, kidney, brain, heart, prostate, lung, breast or liver, histological slides and all possible combinations thereof.

20. Use of a method according to one of the preceding claims for the identification of genes whish are diagnostically relevant for diseases from one of the following categories: cancer diseases; CNS malfunctions, damage or disease; symptoms of aggression or behavioral disturbances; clinical, psychological and social consequences of brain damage; psychotic disturbances and personality disorders; dementia and/or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as a consequence of an abnormality in the development process; malfunction, damage or disease of the skin, the muscles, the connective tissue or the bones; endocrine and metabolic malfunction, damage or disease; headaches or sexual malfunction.

21. Use of a method according to one of the preceding claims for the differentiation of cell types or tissues or for the investigation of cell differentiation.


SEQUENCE LISTING (5 sequences)

SEQ ID NO: 1 - DNA, 18 bp, Artificial Sequence
  bisulfite-treated DNA
ggatttagnggtaagtat

SEQ ID NO: 2 - DNA, 18 bp, Artificial Sequence
  Oligonucleotide
nnnnnnnncgnnnnnnnn

SEQ ID NO: 3 - DNA, 18 bp, Artificial Sequence
  Oligonucleotide
nnnnnnnntgnnnnnnnn

SEQ ID NO: 4 - DNA, 18 bp, Artificial Sequence
  bisulfite-treated DNA
ggatttagnggtaagtat

SEQ ID NO: 5 - DNA, 18 bp, Artificial Sequence
  bisulfite-treated DNA
ggatttagtggtaagtat